(19) United States
(12) Patent Application Publication
(10) Pub. No.: US 2007/0259021 A1
(43) Pub. Date: Nov. 8, 2007

(54) Compositions, Methods, and Kits for Treating Dry Eye

(76) Inventors: Mitchell H. Friedlaender, La Jolla, CA (US); Harun Takruri, Newport Beach, CA (US)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 11/742,924

(22) Filed: May 1, 2007

Related U.S. Application Data
(60) Provisional application No. 60/796,199, filed on May 1, 2006.

(51) Int. Cl. A61K 38/37; A61K 31/717; A61K 38/39

(52) U.S. Cl. 424/427; 424/78.37; 424/78.38; 514/55; 514/57; 514/58; 514/54; 514/2

(57) ABSTRACT

The present invention is related to a method for treating dry eye, the method comprising administering to a lacrimal punctum a gelatinous or film forming composition. The present invention is also related to ophthalmic compositions, and kits for treating dry eye, the kit comprising (a) the ophthalmic compositions of the present invention; and (b) instructions for using the composition of (a) to treat dry eye.

This application claims the benefit of the filing date of U.S. Application No. 60/796,199, filed May 1, 2006, which is incorporated by reference herein in its entirety.

### BACKGROUND OF THE INVENTION

1. Field of the Invention

The present invention is related to a method for treating dry eye in a subject, the method comprising administering to a lacrimal punctum of the subject a gelatinous or film forming composition. The present invention is also related to ophthalmic compositions, and kits for treating dry eye, the kit comprising (a) the ophthalmic compositions of the present invention; and (b) instructions for using the composition of (a) to treat dry eye.

2. Background Art

Dry eye, also known as keratitis sicca or keratoconjunctivitis sicca, can occur when a subject's tear glands produce less tears than normal. This reduced tear level results in symptoms that range from mild irritation and discomfort to severe discomfort and light sensitivity.

Dry eye can be caused by a variety of factors, e.g., aging, autoimmune diseases such as rheumatoid arthritis and lupus, injury, medication, laser vision correction, and environmental factors. Regardless of the cause, ophthalmic solutions, commonly referred to as “artificial tears,” and ointments and gels, commonly referred to as lubricants, are often used for treatment. These artificial tears and lubricants require frequent instillation to the affected eye to prevent drying and discomfort.

An alternative to artificial tears is the use of silicone punctal plugs to treat dry eye. Silicone punctal plugs can be inserted in the tear drainage duct(s) to prevent drainage of tears into the nasolacrimal system. However, several disadvantages to these silicone plugs exist. For example, silicone punctal plugs require insertion and removal by a medical or veterinary professional, e.g., a physician, usually in an office environment. Inconvenience to patients, as well as the clinical costs associated with insertion and removal, reduce the routine and widespread use of these plugs. Silicone punctal plugs can be difficult, or impossible, to insert. In addition, they can have a high fallout rate, or they may be placed deliberately, or inadvertently, in the lacrimal canaliculus, where they can cause inflammation or infection.

A need exists in the art for alternative methods of treating dry eye. For example, a need exists for a method of treating dry eye that reduces the frequency of application of artificial tears. Additionally, a need exists for a method of treating dry eye that does not require a visit to a physician or veterinarian for insertion and removal of the punctal plugs.

### BRIEF SUMMARY OF THE INVENTION

The present invention is related to a method for treating dry eye in a subject, the method comprising administering to a lacrimal punctum of the subject a gelatinous or film forming composition.

In some embodiments, the gelatinous or film forming composition comprises petrolatum. In some embodiments, the composition further comprises a stiffening agent, e.g., beeswax, paraffin, stearic acid, stearyl alcohol, cetyl alcohol, lanolin or lanolin alcohol.

The composition of the present invention can further comprise a mucoadhesive polymer. The mucoadhesive polymer can be selected from the group consisting of polyvinyl alcohol, povidone, water soluble cellulose derivatives, gelatin, natural gums, carbomer polymers, polyacrylates, chondroitin sulfate and salts thereof, hyaluronic acid and salts thereof, dextrans, chitosans, albumins, soluble starches and combinations thereof. In some embodiments, the mucoadhesive polymer is about 0.05% to about 70%, about 0.5% to about 50%, or about 1% to about 25% by weight of the total composition.

In some embodiments of the present invention, the gelatinous or film forming composition does not contain petrolatum. In some embodiments, the gelatinous or film forming composition does not contain a stiffening agent. The gelatinous or film forming composition can comprise polyethylene glycol. In some embodiments, the gelatinous or film forming composition comprises polyvinyl alcohol, povidone, water soluble cellulose derivatives, gelatin, natural gums, carbomer polymers, polyacrylates, chondroitin sulfate and salts thereof, hyaluronic acid and salts thereof, dextrans, chitosans, albumins, soluble starches, or combinations thereof.

The present invention is also related to a method comprising administering to a lacrimal punctum a film forming composition. In some embodiments, the film forming composition hydrates upon administration to the inferior punctum. Alternatively, the film forming composition can be hydrated before being administered to the inferior punctum. In some embodiments, the film forming composition is a laminate. In some embodiments, the laminate comprises a backing composition. For example, the backing composition can be a wax composition or a paper composition.

The present invention is also related to a composition comprising (a) petrolatum; (b) a stiffening agent; and (c) a mucoadhesive polymer, wherein the composition is ophthalmically acceptable. Alternatively, the composition can comprise: (a) petrolatum; (b) beeswax; and (c) a preservative, wherein the ophthalmic composition is ophthalmically acceptable. In another embodiment, the invention is related to a composition comprising: (a) polyethylene glycol; (b) cetostearyl alcohol; and (c) a preservative, wherein the composition is ophthalmically acceptable.

The present invention is also related to a kit for treating dry eye, the kit comprising (a) the gelatinous or film forming composition of the present invention; and (b) instructions for using the composition of (a) to treat dry eye. In some embodiments, the present invention is directed to a kit for treating dry eye, the kit comprising (a) the ophthalmic composition of the present invention; wherein the ophthalmic composition is individually packaged for a single administration; and (b) instructions for using the composition of (a) to treat dry eye.

### DETAILED DESCRIPTION OF THE INVENTION

The present invention is directed to a method for treating dry eye in a subject, the method comprising administering to a lacrimal punctum of the subject a gelatinous or film forming composition. In some embodiments, the gelatinous or film forming composition “plugs” or otherwise impedes the drainage of tears, either natural or artificial, through the punctum, thus maintaining a wet ocular surface. In some embodiments, the gelatinous or film forming composition completely impedes the drainage of tears through the punctum.

The term “dry eye” refers to a condition wherein tear glands of a subject produce less tears than normal. The term “dry eye” is meant to include, but is not limited to, the following related conditions: tear deficiency or insufficiency, tear film deficiency or insufficiency, keratitis sicca, keratoconjunctivitis sicca, and Sjogren's Syndrome. The term “dry eye” can also refer to the drying and inflammation of the conjunctiva as a result of insufficient tear production. The term “treating dry eye” can also refer to the treatment of other conditions which may result in a reduced production of tears. For example, the term “treating dry eye” would include treatment of dry eyes associated with Sjogren's syndrome. The term “tears” refers to the secretions of the lacrimal glands, and accessory lacrimal glands, combined with secretions of the meibomian glands and conjunctival goblet cells. These tears moisten the ocular surface. In many instances, “tears” are slightly alkaline or acidic.

The terms “treating”, “treatment” or “treats” refer to the administering to a subject a composition of the present invention for purposes which can include prevention, amelioration, or cure of dry eye, or the symptoms thereof.

The term “lacrimal punctum” or “puncta” refers to the tear drainage duct system of the eyelid. Thus, as used herein the terms “lacrimal punctum” or “lacrimal puncta” include one or more of the superior puncta or inferior puncta, lacrimal canals, vertical canaliculus, horizontal canaliculus, nasolacrimal sac, and nasolacrimal duct. The term “administer to the lacrimal punctum” refers to the placement of the gelatinous or film-forming composition on, in, or proximate to the lacrimal punctum in an amount sufficient to stop, impede, or reduce the drainage of tears through the lacrimal punctum. One of skill in the art will recognize that administration to the lacrimal punctum can also include depositing some of the gelatinous or film-forming composition to areas proximate to the lacrimal punctum, e.g., the eyelid, the eyelid margin, or the conjunctiva.

Various amounts of the gelatinous or film forming composition of the present invention can be used according to the present invention. In some embodiments, about 10 μl to about 1 ml, about 20 μl to about 500 μl or about 30 μl to about 250 μl of the gelatinous or film forming composition is administered to the puncta in each eye. One of skill in the art will realize that not all of the composition will be administered directly to the puncta, but may be administered near the puncta, e.g., on the areas proximate to the puncta, and achieve similar results. In some embodiments, the composition is administered on an “as desired” basis, i.e., when the subject desires relief from the symptoms of dry eyes. In some embodiments, the composition is administered from 1 to 20 times daily, or from 2 to 12 times daily.

The term “gelatinous composition” refers to a composition having a physical state similar to gelatin or a viscous liquid with significant adhesive properties. The term “gelatinous” can refer to a gel, jelly, ointment, cream, paste or viscous liquid. In some embodiments, the gelatinous composition of the present invention has a viscosity of about 300 centipoise (cps) to about 150,000 cps, about 600 cps to about 100,000 cps, about 1,000 to about 50,000 cps, about 2,000 cps to about 25,000 cps, or about 5,000 cps to about 15,000 cps at 25° C. In some embodiments, the gelatinous composition of the present invention has a viscosity of about 300 cps to about 150,000 cps, about 800 cps to about 100,000 cps, about 1,000 cps to about 50,000 cps, about 2,000 cps to about 25,000 cps, or about 5,000 cps to about 15,000 cps at 37° C. The term “film-forming” composition refers to a composition that begins as a liquid or semi-liquid, that can be placed in a cast and dried to form a solid, semi-solid or glass thin layer or sheet. Once administered to a subject, the film-forming composition can remain as solid, semi-solid or glass thin layer or sheet, or, alternatively, can hydrate to form a gel or viscous liquid.

In some embodiments, the gelatinous or film forming composition comprises petrolatum. “Petrolatum” refers to a substance which is a complex combination of semi-solid, saturated hydrocarbons, mainly of a paraffinic nature, obtained from petroleum. Generally, petrolatum comprises liquid hydrocarbons having carbon numbers predominately greater than C₂₅. Petrolatum can refer to, but is not limited to, a composition having a CAS number 8009-03-8. However, petrolatum made by other processes and thus having slightly different purities and refinements are also within the scope of the term “petrolatum.” In some embodiments, the gelatinous or film forming composition is substantially nonaqueous. In some embodiments, the gelatinous or film forming agent can be biodegradable. Alternatively, in some embodiments the gelatinous or film forming agent is non-biodegradable.

In the present invention, the gelatinous or film-forming composition can comprise a stiffening agent. Various stiffening agents can be used. Generally, these stiffening agents, when combined with petrolatum, raise the viscosity and/or melting point of the resulting mixture. For example, in some embodiments addition of a stiffening agent to a gelatinous composition will increase the viscosity of the resulting mixture such that it is more easily handled for administration. In some embodiments, the addition of a stiffening agent to a gelatinous composition increases the viscosity of the resulting mixture such that the mixture remains viscous (i.e., a viscosity above 300 cps, 800 cps, 1,000 cps, 2,000 cps, or 5,000 cps) even at temperature at or above the temperatures on the punctal surface (i.e., above 37° C. when placed on a human). In some embodiments, the stiffening agent raises the melting point of the resulting mixture to above 40° C., 45° C., or 50° C. In some embodiments, the increased melting point increases adherence to the punctum, thus prolonging the drainage-blocking action of the composition relative to compositions not comprising a stiffening agent. Stiffening agents can include, but are not limited to, beeswax, paraffin, stearic acid, stearyl alcohol, cetyl alcohol, lanolin and lanolin alcohol.

Various amounts of stiffening agents can be used in the composition used in the present invention. In some embodiments, the stiffening agent is about 1% to about 50% w/w of the total compositions, or about 5% to about 30% w/w of the total composition, or about 10% to about 20% w/w of the total composition. In some embodiments, the stiffening agent is present in an amount suitable to increase the viscosity of the composition so that the composition can impede, stop, or reduce the flow of tears through the lacrimal puncta. For example, in some embodiments, the stiffening agent can increase the viscosity of the composition from about 500 cps to about 150,000 cps, about 1,000 cps to about 100,000 cps, about 2,000 cps to about 100,000 cps, about 5,000 cps to about 75,000 cps, about 10,000 cps to about 50,000 cps, about 15,000 cps to about 50,000 cps, about 20,000 cps to about 50,000 cps, or about 25,000 cps to about 50,000 cps, at 25° C. or alternatively at 37° C.

In some embodiments of the present invention, the composition further comprises a mucoadhesive polymer. A mucoadhesive polymer is any polymer that increases adhesion to the ocular surface, lacrimal punctum, or other associated ocular structures. The mucoadhesive polymer can be selected from the group consisting of polyvinyl alcohol, povidone, water soluble cellulose derivatives, gelatin, natural gums, carbomer polymers, polyacrylates, chondroitin sulfate and salts thereof, hyaluronic acid and salts thereof, dextrans, chitosans, albumins, soluble starches and combinations thereof. Various concentrations of the mucoadhesive can be present in the composition of the present invention. For example, the mucoadhesive polymers can be about 0.05% to about 70%, about 0.5% to about 50%, or about 1% to about 25% w/w of the total composition.

In some embodiments, the gelatinous or film forming composition does not contain petrolatum. For example, the gelatinous or film forming composition can comprise polyethylene glycol instead of petrolatum. Polyethylene glycol (PEG) is a condensation polymer of ethylene glycol having the formula HOCH₂(CH₂OCH₂)ₙCH₂OH, wherein n is the average number of oxyethylene groups. The value of n can vary. In some embodiments, the value of n is 2 to 210. In some embodiments, the value of n is 4 to 180. A combination of PEGs having various molecular weights can be used. In some embodiments, a PEG is used which has a melting point above 30° C., above 35° C., above 37° C., or above 40° C. In some embodiments, the gelatinous or film forming composition does not comprise a carboxy vinyl polymer.

In some embodiments, the gelatinous or film forming composition does not contain petrolatum or a stiffening agent. For example, in some embodiments, the gelatinous or film forming composition does not contain petrolatum or a stiffening agent, but does comprise polyvinyl alcohol, povidone, water soluble cellulose derivatives, gelatin, natural gums, carbomer polymers, polyacrylates, chondroitin sulfate or salts thereof, hyaluronic acid or salts thereof, dextrans, chitosans, albumins, soluble starches, or combinations thereof. In some embodiments, the petrolatum-free, stiffening agent-free gelatinous or film forming composition contains a preservative such as, but not limited to, benzalkonium chloride.

In some embodiments, the gelatinous or film forming composition comprises a crosslinked arcylic acid polymer. For example, in some embodiments, the polymer comprises lightly crosslinked acrylic acid polymers wherein the crosslinking monomer is 2,3-dihydroxyhexa-1,5-diene or 2,3-dimethylhexa-1,5-diene. Commercially available polymers include carbopol, and polycarbophil, e.g., the carboxy-containing polymer system known by the tradename DuraSite® (InSite Vision, Inc., Alameda, Calif.).

The present invention is also directed to a method for treating dry eye, comprising administering to a lacrimal punctum a film forming composition as described herein. In some embodiments, the film forming composition hydrates, or takes up other liquid, e.g., tears, upon administration to the lacrimal punctum, whereby the composition forms a viscous mucoadhesive film that seals the punctum and inhibits, impedes, reduces, and/or delays, tear drainage. Alternatively, the film forming composition can be hydrated before being administered to the lacrimal punctum, e.g., with water or a saline solution.

Various film forming compositions can be used. In some embodiments, the film forming composition is a laminate. The term laminate refers to two or more layers wherein the faces of the layers are proximate and adjacent to each other. In some embodiments, the laminate contains a film layer of the present invention and a backing composition. In some embodiments, the laminate can contain two or more layers of the present invention and a backing composition. Various backing compositions can be used. Examples of backing compositions include, but are not limited to a wax composition or a paper composition. In some embodiments, the backing composition can be removed after administration to the punctum. Alternatively, in some embodiments, the backing composition is removed prior to administration to the punctum, e.g., immediately preceding administration to the punctum.

The present invention is also related to an ophthalmic composition comprising (a) petrolatum; (b) a stiffening agent; and (c) a mucoadhesive polymer, wherein the ophthalmic composition is ophthalmically acceptable. Alternatively, the ophthalmic composition comprises: (a) petrolatum; (b) beeswax; and (c) a preservative, wherein the ophthalmic composition is ophthalmically acceptable. In another embodiment, the invention is related to an ophthalmic composition comprising: (a) polyethylene glycol; (b) cetostearyl alcohol; and (c) a preservative, wherein the ophthalmic composition is ophthalmically acceptable.

As used herein, the term “ophthalmically acceptable” refers to those compounds, compositions, and/or solutions which are, within the scope of sound medical judgment, suitable specifically for contact with the tissues of they eye, and the area surrounding the eye without excessive toxicity, irritation, allergic response, or other problem complications commensurate with a reasonable benefit/risk ratio. The compositions of the present invention as described herein are ophthalmically acceptable.

The compositions of the present invention can be administered to a subject in need thereof. As used herein, “subject” refers to any animal which contains a lacrimal punctum, or equivalent structure. Animals can include humans and nonhumans, such as but not limited to, domestic and farm animals, zoo animals, sport animals and pets. In some aspects of the present invention, the subject is a human. In some aspects of the present invention, the subject is a human adult. In some embodiments, the present invention is directed to a method of treating dry eye, the method comprising administering an effective amount of the composition of the present invention to a subject in need thereof. One advantage of the present invention is that it may be self administered by the subject. Another advantage of the present invention is that it may be administered by a non-medical or non-veterinarian person, e.g., a parent can administer the composition to a child, or a pet owner can administer it to a pet, without the assistance of a physician or a veterinarian.

The present invention is also related to a kit for treating dry eye. The kit can comprise (a) the gelatinous or film forming composition of the present invention; and (b) instructions for using the composition of (a) to treat dry eye. In some embodiments, the present invention is directed to a kit for treating dry eye, the kit comprising (a) the gelatinous or film forming composition of the present invention; wherein the composition is individually packaged for a single administration; and (b) instructions for using the composition of (a) to treat dry eye.

The instructions for using the composition can be in a form prescribed by a governmental agency regulating the manufacture of the composition, use or sale of the composition, or use or sale for human application. In some embodiments, the kit can comprise instructions, which, e.g., provide information on the use of the pharmaceutical composition. In some embodiments, the instructions can be a pre-recorded media device which, e.g., provides information on the use of the method of the present invention.

The instructions can be in the form of printed matter. “Printed matter” can be, for example, a book, booklet, brochure, leaflet or the like. The printed matter can describe the use of the pharmaceutical composition of the present invention. Possible formats include, but are not limited to, a bullet point list, a list of frequently asked questions (FAQ) or a chart. Additionally, the information to be imparted can be illustrated in non-textual terms using pictures, graphics or other symbols.

The kit can also include a container for storing the components of the kit. The container can be, for example, a bag, box, envelope or any other container that would be suitable for use in the present invention. In some embodiments, the container is large enough to accommodate each component of the kit of the present invention. However, in some cases, it can be desirable to have a smaller container which is large enough to carry only some of the components of the present invention.

In some embodiments, the present invention contains individual packages that hold an amount of the composition sufficient for a single application. The individual packages can be in various forms, including but not limited to, bottles, jars, ampoules, tubes, syringes, or envelopes. Alternatively, the present invention can contain packages that hold an amount of the composition sufficient for two or more applications. For example, the package can contain an amount of the composition sufficient for 2 to 100 administrations, or 2 to 30 administrations, 2 to 14 administrations, or 2 to 7 administrations. The package can be a resealable package. In some embodiments, the package can further comprise a dosage dispenser which can dispense an appropriate amount of the composition for use in a single application. The package which contains two or more applications can be in various forms, including, but not limited to bottles, jars, ampoules, tubes, syringes, or envelopes.

Various means can be used to apply the gelatinous or film forming composition to the lacrimal punctum. In some embodiments, the composition can be applied using a finger. In some embodiments, a delivery device such as, but not limited to, a terry cloth pad, a cotton pad, a plastic dispenser, or a paper strip can be used to apply the gelatinous or film forming composition. An example of a delivery device can be a filter strip paper, similar to a Schirmer strip used in measuring tear production. In one example, one end of the filter paper strip can be coated with the composition of the present invention. The coated end of the filter paper strip is then placed near the inner canthal region (between the eye and the bridge of the nose), wherein the composition of the invention is released (applied), flowing onto the adjacent structures, including the lacrimal puncta. A drop of liquid, such as an artificial tear or saline, can be used to wet the strip in order to mobilize the coated material.

### EXAMPLES

### Example 1

A composition containing petrolatum and white beeswax was made as described in Table 1:

TABLE 1

| Ingredient | % w/w |
|---|---|
| White Beeswax | 20.0 |
| White Petrolatum | 79.5 |
| Chlorobutanol | 0.5 |

The ingredients of Table 1 were melted in a water bath and mixed to homogeneity. The mixture was cooled while mixing until the mixture congealed and was then packaged into appropriate containers.

### Example 2

A composition containing petrolatum and cetostearyl alcohol was made as described in Table 2:

TABLE 2

| Ingredient | % w/w |
|---|---|
| Cetostearyl Alcohol | 20.0 |
| White Petrolatum | 79.5 |
| Chlorobutanol | 0.5 |

The ingredients of Table 2 were melted in a water bath and mixed to homogeneity. The mixture was cooled while mixing until the mixture congealed and was then packaged into appropriate containers.

### Example 3

A composition containing polyethylene glycol, cetostearyl alcohol, and chlorobutanol was made as described in Table 3:

TABLE 3

| Ingredient | % w/w |
|---|---|
| PEG 600 | 65.0 |
| PEG 3350 | 30.0 |
| Cetostearyl Alcohol | 4.5 |
| Chlorobutanol | 0.5 |

The ingredients of Table 3 were melted in a water bath and mixed to homogeneity. The mixture was cooled while mixing until the mixture congealed and was then packaged into appropriate containers.

### Example 4

A composition containing carbomer, carboxymethylcellulose sodium, povidone, benzalkonium chloride and water was made as described in Table 4:

TABLE 4

| Ingredient | % w/w |
|---|---|
| Carbomer (Carbopol 980) | 4.0 |
| Carboxymethylcellulose Sodium | 0.5 |
| Povidone (Kollidon 90) | 1.0 |
| Benzalkonium Chloride | 0.01 |
| Sodium Hydroxide | to adjust to pH 5-7 |
| Water | q.s. 100 grams |

The carbomer was dispersed in a portion of the water and adjusted to a pH of about 6.0 with sodium hydroxide to form a carbomer dispersion. The remaining components were then dissolved in another portion of water. Once the remaining components were dissolved, they were added to the carbomer dispersion and mixed to yield a uniform adhesive gel.

### Example 5

A composition containing carbomer, benzalkonium chloride and maple syrup was made as described in Table 5:

TABLE 5

| Ingredient | % w/w |
|---|---|
| Carbomer (Carbopol 980), 8% gel | 40.0 |
| Benzalkonium Chloride, 0.5% | 2.0 |
| Maple syrup (grade A medium amber, 100% pure) | q.s. 100 grams |
| Carbomer (Carbopol 980), 8% gel | 40.0 |

The components of Table 5 were mixed together to homogeneity to yield a uniform adhesive gel.

### Example 6

A composition containing polyvinyl alcohol, glycerin, hypromellose, gelatin, and benzalkonium chloride was made as described in Table 6:

TABLE 6

| Ingredient | % w/w |
|---|---|
| Polyvinyl Alcohol | 10.0 |
| Glycerin | 10.0 |
| Hypromellose (K100M) | 4.0 |
| Gelatin | 1.0 |
| Benzalkonium Chloride | 0.01 |
| Water | q.s. 100 ml |

The polyvinyl alcohol and gelatin were dissolved in a heated mixture of water and glycerin at about 90° C. The hypromellose was dispersed at the high temperature and cooled while mixing to dissolve the hypromellose. Benzalkonium chloride was added to the cooled gel and mixed to yield a uniform adhesive gel.

### Example 7

The adhesive gel in Example 6 was cast on a glass plate and placed in an oven until dry and easy to manipulate. The dried film was then cut into small squares suitable for application to the punctum.

It is to be appreciated that the Detailed Description section, and not the Summary and Abstract sections, is intended to be used to interpret the claims. These examples illustrate possible compositions used in the present invention. While the invention has been particularly shown and described with reference to some embodiments thereof, it will be understood by those skilled in the art that they have been presented by way of example only, and not limitation, and various changes in form and details can be made therein without departing from the spirit and scope of the invention. Thus, the breadth and scope of the present invention should not be limited by any of the above-described exemplary embodiments, but should be defined only in accordance with the following claims and their equivalents.

All documents cited herein, including journal articles or abstracts, published or corresponding U.S. or foreign patent applications, issued or foreign patents, or any other documents, are each entirely incorporated by reference herein, including all data, tables, figures, and text presented in the cited documents.

What is claimed is:

1. A method for treating dry eye in a subject, the method comprising administering to a lacrimal punctum of the subject a gelatinous or film forming composition.

2. The method of claim 1, wherein the gelatinous or film forming composition comprises petrolatum.

3. The method of claim 1, the composition further comprising a stiffening agent.

4. The method of claim 3, wherein the stiffening agent is selected from beeswax, paraffin, stearic acid, stearyl alcohol, cetyl alcohol, lanolin and lanolin alcohol.

5. The method of claim 2, the composition further comprising a mucoadhesive polymer.

6. The method of claim 5, wherein the mucoadhesive polymer is selected from polyvinyl alcohol, povidone, water soluble cellulose derivatives, gelatin, natural gums, carbomer polymers, polyacrylates, chondroitin sulfate and salts thereof, hyaluronic acid and salts thereof, dextrans, chitosans, albumins, soluble starches, and combinations thereof.

7. The method of claim 5, where the mucoadhesive polymer is about 0.5% to about 50% w/w of the total composition.

8. The method of claim 1, wherein the gelatinous or film forming composition comprises polyethylene glycol.

9. The method of claim 1, wherein the gelatinous or film forming composition does not contain petrolatum.

10. The method of claim 1, wherein the gelatinous or film forming composition does not contain a stiffening agent.

11. The method of claim 10, wherein the gelatinous or film forming composition comprises polyvinyl alcohol, povidone, water soluble cellulose derivatives, gelatin, natural gums, carbomer polymers, polyacrylates, chondroitin sulfate and salts thereof, hyaluronic acid and salts thereof, dextrans, chitosans, albumins, soluble starches, or combinations thereof.

12. The method of claim 1, the method comprising administering to a lacrimal punctum a film forming composition.

13. The method of claim 12, wherein the film forming composition hydrates upon administration to the inferior punctum.

14. The method of claim 12, wherein the film forming composition is hydrated before being administered to the inferior punctum.

15. The method of claim 12, where the film forming composition is a laminate.

16. The method of claim 15, wherein the laminate comprises a backing composition.

17. The method of claim 16, wherein the backing composition is a wax composition or a paper composition.

18. An ophthalmic composition comprising
(a) petrolatum;
(b) a stiffening agent; and
(c) a mucoadhesive polymer,
wherein the ophthalmic composition is ophthalmically acceptable.

19. An ophthalmic composition comprising:
(a) petrolatum;
(b) beeswax; and
(c) a preservative,
wherein the ophthalmic composition is ophthalmically acceptable.

20. An ophthalmic composition comprising:
(a) polyethylene glycol;
(b) cetostearyl alcohol; and
(c) a preservative,
wherein the ophthalmic composition is ophthalmically acceptable.

21. A kit for treating dry eye, the kit comprising
(a) the ophthalmic composition of claim 1; and
(b) instructions for using the composition of (a) to treat dry eye.

22. A kit for treating dry eye, the kit comprising
(a) the ophthalmic composition of claim 1; wherein the ophthalmic composition is individually packaged for a single administration; and
(b) instructions for using the composition of (a) to treat dry eye.
